(19) United States
(12) Patent Application Publication
(10) Pub. No.: US 2024/0142373 A1
(43) Pub. Date: May 2, 2024

(54) PERACETIC ACID SENSING LAYER COMPOSITION AND RELATED SYSTEMS

(71) Applicant: Georgia Tech Research Corporation, Atlanta, GA (US)

(72) Inventors: Jie XU, Marietta, GA (US); Walker Thomas Byrnes, Atlanta, GA (US)

(21) Appl. No.: 18/385,106

(22) Filed: Oct. 30, 2023

Related U.S. Application Data
(60) Provisional application No. 63/420,363, filed on Oct. 28, 2022.

(51) Int. Cl. G01N 21/45; G01N 33/53

(52) U.S. Cl. CPC G01N 21/45; G01N 33/5308

(57) ABSTRACT

A sensing layer composition is provided. The sensing layer composition is particularly suited to be adhered to at least one side of one or more waveguide channels in/on a waveguide chip of an interferometric system. The sensing layer composition is particularly suited to bind or be selectively disturbed by peracetic acid via optical interferometric analysis.

### CROSS-REFERENCE TO RELATED APPLICATIONS

The present application claims priority to U.S. Provisional Application No. 63/420,363, filed Oct. 28, 2022, the content of which is incorporated herein by reference.

### BACKGROUND

Peracetic acid (also known as peroxyacetic acid or PAN is an organic peroxide that is typically colorless with a pungent vinegar-like odor. Peracetic acid may be formed from the reaction of acetic acid and hydrogen peroxide. Peracetic acid is highly corrosive and may irritate and burn the lungs, throat, skin or eyes if not handled appropriately. Historically, peracetic acid has been used as a disinfectant in diverse applications, including food processing, medical, chemical, and pulp/paper industries due to its wide spectrum antimicrobial activity. Peracetic acid can inactivate gram-positive and gram-negative bacteria, fungi, and yeasts.

Commercially available peracetic acid is often a mixture of H₂O₂, acetic acid, and water with a peracetic acid content of 5% to 40% (wt. %). The effectiveness of peracetic acid as a disinfectant depends on its concentration, contact time, the susceptibilities of the target organisms, and organic loads in the matrix of the material being disinfected. Peracetic acid may be decomposed in aqueous solution into oxygen, hydrogen peroxide, and acetic acid via spontaneous decomposition, hydrolysis, and transition metal based catalysis. The decomposition rate is highly depended on the pH, temperature, and the level of transition metals. Additionally, peracetic acid is consumed rapidly in aqueous solutions due to the presence of materials and living organisms that generate chemical oxygen demand (COD) and biological oxygen demand (BOD). The decay of peracetic acid due to oxidant demand may be rapid, occurring within the first few minutes of contact time. Controlling the rate of decays is an important economic concern.

Methods have been reported to measure the concentration of peracetic acid including chromatographic, potentiometric, titrimetric, and colorimetric. Field employed practices based on colorimetric method and redox titration can be impeded by the presence of color and turbidity in the process water. These practices require manual sample collection and measurement. Several peracetic acid sensors have been developed including commercial amperometric sensors and biosensors. Frequent sensor calibration may be needed and electrode fouling is often observed.

### SUMMARY

The present disclosure provides improved systems and methods to enable management of peracetic acid loading in various environments. The present systems and methods are based on a different technology that provide a higher target sampling frequency compared to existing technology, and automate what is normally an arduous manual process.

A sensing layer composition is provided. The sensing layer composition is sensitive to and may selectively bind to one or more analytes. The sensing layer composition may also reversibly release one or more analytes. The sensing layer composition may be adhered to at least one side of one or more waveguide channels in/on a waveguide chip of an interferometric system (e.g., optical interferometric system). According to a particular embodiment, the sensing layer composition is adapted to bind one or more analytes that are detected via interferometric analysis. According to a particular embodiment, the sensing layer composition is configured to selectively bind one or more analytes in a liquid buffer. According to a particular embodiment, the sensing layer composition is configured to selectively bind one or more analytes in a buffer that includes acetic acid and hydrogen peroxide without interference from the acetic acid and hydrogen peroxide.

According to one embodiment, the sensing layer composition is a charge-transfer based sensing layer composition. According to one embodiment, the sensing layer composition is formulated as a charge-transfer based sensing film. According to one embodiment, the sensing layer composition include iodine. According to one embodiment, the sensing layer composition is formulated as an iodine-containing film. According to one embodiment, the sensing layer composition includes a charge transfer complex formed between iodine (electron acceptor) and at least one aromatic hydrocarbon such an aromatic polymer. Aromatic polymers containing electron donating functional groups include, but are not limited to, phenol, styrene, or a combination thereof.

According to one embodiment, the charge transfer complex may be dissociated so the sensing layer composition can be used more than one time or continuously (i.e., used to selectively bind to and reversibly release one or more analytes). According to one embodiment, the sensing layer composition that is utilized more than once includes styrene as the aromatic polymer.

According to one embodiment, the sensing layer composition is configured in an interferometric system to sense peracetic acid in an optical interferometer in situ. According to one embodiment, the sensing layer composition is configured in an interferometric system as provided herein to sense or otherwise monitor levels of peracetic acid in a target sample in situ so as to provide real-time or near real-time data regarding peracetic acid presence and peracetic acid quantity within a target sample.

An analyte sensor system is also provided. According to one embodiment, the analyte sensor system includes a target sample handing system and an interferometric system as provided herein. According to one embodiment, the target sample handling system is upstream of the interferometric system. According to such an embodiment, the target sample handing system includes an automated flow injection system. The automated flow injection system may include various components that provide end-to-end automation of the target sample collection and interferometric system introduction. According to a particular embodiment, the automated flow injection system may include various components that perform one or more functions such as collecting process liquid including target sample (such as sample stream of water from a chiller), changing the concentration of the target sample including, for example, performing a dilution (including an in situ dilution), and delivering the diluted target sample to the interferometric system.

### BRIEF DESCRIPTION OF THE DRAWINGS

FIG. 1 is a schematic of a target sample handling system and interferometric system according to one embodiment.

FIG. 2 illustrates a method of detecting and quantifying the level of one or more analytes such as peracetic acid according to one embodiment.

### DETAILED DESCRIPTION

One or more aspects and embodiments may be incorporated in a different embodiment although not specifically described. That is, all aspects and embodiments can be combined in any way or combination. When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

### Definitions

As used herein, the term “portable” refers to the capability of the interferometric systems described herein to be transported or otherwise carried to a target sample location for use according the methods provided herein.

As used herein, the term “analyte” refers to a substance that is detected, identified, measured or any combination thereof by the systems provided herein. The analyte includes any solid, liquid, or gas within an environment of interest or that is targeted. The analyte includes, but is not limited to, peracetic acid.

As used herein, the term “iodine” generally refers to the known trace element (I) that is the oxidation product produced between the interaction of peracetic acid and iodide.

As used herein, the terms “sample” and “target sample” all refer to any substance that may be subject to the methods and systems provided herein. Particularly, these terms refer to any matter (animate or inanimate) where one or more analytes may be present and capable of being detected, quantified, monitored or a combination thereof in a batch or continuous manner. Suitable examples of targets include, but are not limited to, liquids such as waters used in food processing for temperature control of the food or disinfection of equipment, any animate or inanimate surface, water or water source (e.g., drinking water source), soil, food, ambient air, soil, cleaning products (disinfectants, sterilants, sanitizers, oxidants), reagents, polymerization catalysts, fabrics, grease-resistant paper, cookware, personal products, stain-resistant coatings, aquatic animals (e.g., fish), fire retardants, bodily fluids (e.g., blood, breast milk, spinal fluid, cord blood, saliva, or amniotic fluid), agricultural sites, and landfills. Target samples may be taken from air, surfaces, fluids and mixtures thereof in or from manufacturing or processing facilities (e.g., food or pulp/paper manufacturing facilities), water treatment plants, laboratories, farms, healthcare facilities, wineries/breweries or laundry facilities.

As used herein, the term “point of use” refers to the applicability of the systems provided herein to be utilized by a user at or in a particular environment (e.g., on site).

“Optional” or “optionally” means that the subsequently described event or circumstance may or may not occur, and that the description includes instances where said event or circumstance occurs and instances where it does not.

As used herein, the term “buffer” refers to a carrier that is mixed with the target sample that includes at least one analyte. The buffer may contain compounds designed to moderate change in chemical properties. The buffer may also include one or more anti-foam compounds. An exemplary buffer includes, but is not limited to, potassium iodide.

As used herein, the term “test sample composition” refers to target sample and, optionally, at least one buffer.

As used herein, the term “communication” refers to the movement of air, liquid, mist, fog, buffer, test sample composition, or other suitable source capable of carrying an analyte throughout or within the cartridge system. The term “communication” may also refer to the movement of electronic signals between components of the systems described herein.

As used herein, the term “single-use” refers to the cartridge system being utilized in an interferometric system for a single test or assay before disposal (i.e., not re-used or used for a second time).

As used herein, the term “multiple-use” refers to a cartridge system being utilized for more than one test sample composition (e.g., assay) before disposal.

As used herein, the term “multiplex” refers to a cartridge system being utilized to detect multiple analytes from one target sample composition.

### Optical Interferometry Principles

The sensing layer compositions provided herein may be utilized in various interferometric systems. According to a particular embodiment, such systems include a detector that operates via ultrasensitive, optical waveguide interferometry. The waveguiding and the interferometry techniques are combined to detect, monitor and even measure small changes that occur in an optical beam along a propagation pathway. These changes can result from changes in the length of the beam's path, a change in the wavelength of the light, a change in the refractive index of the media the beam is traveling through, or any combination of these, as shown in Equation 1.

φ=2πLn/λ Equation 1

According to Equation 1, φ is the phase change, which is directly proportional to the path length, L, and refractive index, n, and inversely proportional to the wavelength (λ) change. According to the systems and methods provided herein, the change in refractive index is used. Optical waveguides are utilized as efficient sensors for detection of refractive index change by probing near the surface region of the sample with an evanescent field. Particularly, the systems provided herein can detect small changes in an interference pattern.

According to one embodiment, the waveguide and interferometer act independently or in tandem to focus an interferometric diffraction pattern. According to one embodiment, the waveguide, interferometer, and sensor act independently or two parts in tandem, or collectively to focus an interferometric pattern with or without mirrors or other reflective or focal median. According to one embodiment, the waveguide and interferometer exhibit a coupling angle such that focus is at an optimum angle to allow the system to be compact and suited to be portable and hand-held.

### Interferometric Systems

Interferometric systems may utilize the sensing layer composition provided herein. Such interferometric system may be mobile (hand-held) or otherwise portable for ease of use in various environments. The interferometric system may be built into a fixed station as part of a larger installation of production equipment. The interferometric systems may include a weight and overall dimensions such that user may hold the entire interferometric system comfortably in one hand. According to one embodiment, the entire interferometric system lightweight, handheld and easy-to-use interferometric system that can rapidly, precisely, and accurately provide detection and quantification of analytes in a variety of environments.

The interferometric systems as provided herein that may utilize the sensing layer composition provide a high throughput modular design. The systems as provided herein may provide both qualitative and quantitative results from one or more analytes. Particularly, the systems as provided herein may simultaneously provide detection and quantification of one or more analytes from a target sample. According to one embodiment, both qualitative and quantitative results are provided in real-time or near real time.

The interferometric systems as provided herein that may utilize the sensing layer composition can generally include a housing for various detection, analysis and display components. The interferometric system housing includes a rugged, stable, shell or case. The interferometric system housing can withstand hazards of use and cleaning or disinfection procedures of the case surface. The interferometric system housing may be manufactured from a polymer via various techniques such as injection molding or 3D printing. The interferometric system housing may be manufactured to include a coloration that provides the interferometric system housing with a particular color or color scheme.

According to one embodiment, the interferometric systems provided herein include components that are sealed, waterproof or water resistant to the outside environment to minimize opportunities for contamination of a target sample. The overall arrangement of components within the interferometric systems minimize harboring of contamination in any hard-to-reach areas allowing for ease of disinfection.

The interferometric systems provided herein that may utilize the sensing layer composition include a cartridge system. The cartridge systems provided herein integrate with one or more independent or integrated optical waveguide interferometers. The cartridge systems provide efficient sample composition communication through a microfluidic system mounted on or within the cartridge housing. The cartridge is suitable for one or more analytes to be detected in a single sample in a concurrent, simultaneous, sequential or parallel manner. The cartridge systems provided herein may be utilized to analyze in a multiplex manner. That is, one test sample will be tested to determine the presence of multiple analytes at the same time by utilizing a plurality of waveguide channels that interact with the test sample.

The cartridge systems provided herein are easily removable and disposable allowing for overall quick and efficient use without the risk of cross-contamination from a previous target sample. The cartridge may be safely disposed of after a single use. Disposal after a single use may reduce or eliminate user exposure to hazards. According to one embodiment, the cartridge system includes materials that are biodegradable, or recycled materials, to reduce environmental impact. The cartridge system may be cleaned and re-used or otherwise recycled after a single use.

The cartridge system as provided herein may be suited for multiple or one-time use. The single-use cartridge system may be manufactured in a manner such that a buffer solution is pre-loaded in the microfluidic system. By providing the buffer solution pre-loaded in the single-use cartridge system, gas bubbles are reduced or otherwise eliminated. After a single use, the entire cartridge system is safely discarded or recycled for later use after cleaning. Put another way, after introduction and detection of a test sample, the entire single-use cartridge system is not used again and, instead, discarded.

The cartridge systems as provided herein may be suited for multiple uses. According to such an embodiment, the cartridge system may be used one or more times prior to the cartridge system being safely discarded or recycled. The cartridge system may also be cleaned and re-used or otherwise recycled after multiple uses. According to one embodiment, the cartridge system facilitates cleaning and re-tooling to allow the cartridge system to be replenished and returned to operation.

According to one embodiment, the interferometric systems provided herein provide both qualitative and quantitative results at or under 60 minutes after sample introduction to the system. According to one embodiment, both qualitative and quantitative results are provided at or under 30 minutes. According to one embodiment, both qualitative and quantitative results are provided at or under 10 minutes. According to one embodiment, both qualitative and quantitative results are provided at or under 5 minutes. According to one embodiment, both qualitative and quantitative results are provided at or under 2 minutes. According to one embodiment, both qualitative and quantitative results are provided at or under 1 minute.

The interferometric systems as provided herein may be powered via alternating current or direct current. The direct current may be provided by a battery such as, for example, one or more lithium or alkaline batteries. The alternating or direct current may be provided by alternative energy sources such as wind or solar.

According to one embodiment, the interferometric system is stabilized to address vibrational distortions. The system may be stabilized by various means including mechanical, chemically (fluid float or gel pack), computer-assisted system (electronically), or digitally (e.g., via a camera). In some implementations, the systems provided herein allow for point of use assays that are stable in various conditions, including ambient temperature and humidity as well as extreme heat, cold and humidity.

The interferometric systems as provided herein may be equipped with one or more software packages loaded within. The software may be electronically connected to the various system components as provided herein. The software may also be electronically integrated with a display for viewing by a user. The display may be any variety of display types such as, for example, a LED-backlit LCD. The system may further include a video display unit, such as a liquid crystal display (“LCD”), an organic light emitting diode (“OLED”), a flat panel display, a solid state display, or a cathode ray tube (“CRT”).

According to one embodiment, the interferometric system as provided herein may interface with or otherwise communicate with a transmission component. The transmission component may be in electronic signal communication with both the cartridge system and interferometric system components. The transmission component sends or transmits a signal regarding analyte detection data and quantification data. The transmission of such data may include real-time transmission via any of a number of known communication channels, including packet data networks and in any of a number of forms, including instant message, notifications, emails or texts. Such real-time transmission may be sent to a remote destination via a wireless signal thereby allowing real-time or nearly real-time remote monitoring of peracetic acid. The wireless signal may travel via access to the Internet via a surrounding Wi-Fi network. The wireless signal may also communicate with a remote destination via Bluetooth or other radio frequency transmission. The remote destination may be a smart phone, pad, computer, cloud device, or server. The server may store any data for further analysis and later retrieval. The server may analyze any incoming data using artificial intelligence learning algorithms or specialized pathological, physical, or quantum mechanical expertise programed into the server and transmit a signal.

According to one embodiment, the transmission component may include a wireless data link to a phone line. Alternatively, a wireless data link to a building Local Area Network may be used. The system may also be linked to Telephone Base Unit (TBU) which is designed to physically connect to a phone jack and to provide 900 MHz wireless communications thereby allowing the system to communicate at any time the phone line is available.

According to one embodiment, the interferometric system may include a location means. Such a location means includes one or more geolocation device that records and transmits information regarding location. The location means may be in communication with a server, either from a GPS sensor included in the system or a GPS software function capable of generating the location of the system in cooperation with a cellular or other communication network in communication with the system. According to a particular embodiment, the location means such as a geolocation device (such as GPS) may be utilized from within its own device or from a mobile phone or similarly collocated device or network to determine the physical location of the cartridge system.

According to one embodiment, the interferometric system contains a geo-location capability that is activated when a sample is analyzed to “geo-stamp” the sample results for archival purposes. According to one embodiment, the interferometric system contains a time and date capability that is activated when a sample is analyzed to time stamp the sample results for archival purposes.

The interferometric systems provided herein may interface with software that can process the signals hitting the detector unit. The cartridge system as provided herein may include a storage means for storing data. The storage means is located on or within the cartridge housing or within the interferometric system housing. The storage means communicates directly with electronic components of the interferometric system. The storage means is readable by the interferometric system. Data may be stored as a visible code or an index number for later retrieval by a centralized database allowing for updates to the data to be delivered after the manufacture of the cartridge system. The storage means may include memory configured to store data provided herein.

The data retained in the storage means may relate to a variety items useful in the function of the interferometric system. According to a particular embodiment, the data may provide the overall interferometric system or cartridge system status such as whether the cartridge system was previously used or is entirely new or un-used. According to a particular embodiment, the data may provide a cartridge system or interferometric system identification. Such an identification may include any series of letter, numbers, or a combination thereof. Such identification may be readable through a quick response (QR) code. The identification may be alternatively memorialized on a sticker located on the cartridge housing or interferometric system housing. According to one embodiment, the cartridge housing contains a bar code or QR code. According to one embodiment, the cartridge system contains a bar code or QR code for calibration or alignment. According to one embodiment, the cartridge system contains a bar code or QR code for identification of the cartridge or test assay to be performed. According to one embodiment, the cartridge system contains a bar code or QR code for identification of the owner and location of where any data generated should be transmitted. A user may scan such a QR code with the interferometric system's external camera prior to use to use of the system such that identification and transmission may occur (e.g., automatically or upon user direction).

According to a particular embodiment, the data retained in the storage means may provide the number of uses remaining for a multiple-use cartridge system. According to a particular embodiment, the data may provide calibration data required by interferometric system to process any raw data into interpretable results. According to a particular embodiment, such data may relate to information about the analyte and any special processing instructions that can be utilized by the cartridge system to customize the procedure for the specific combination of receptive surface(s) and analyte(s). The interferometric system as provided herein may include electronic memory to store data via a code or an index number for later retrieval by a centralized database allowing for updates to the data to be delivered after the manufacture of the cartridge system.

The interferometric system may include a memory component such that operating instructions for the interferometric system may be stored. All data may be stored or archived for later retrieval or downloading onto a workstation, pad, smartphone or other device. According to one embodiment, any data obtained from the system provided herein may be submitted wirelessly to a remote server. The interferometric system may include logic stored in local memory to interpret the raw data and findings directly, or the system may communicate over a network with a remotely located server to transfer the raw data or findings and request interpretation by logic located at the server. The interferometric system may be configured to translate information into electrical signals or data in a predetermined format and to transmit the electrical signals or data over a wireless (e.g., Bluetooth) or wired connection within the system or to a separate mobile device. The interferometric system may perform some or all of any data adjustment necessary, for example adjustments to the sensed information based on analyte type or age, or may simply pass the data on for transmission to a separate device for display or further processing.

The interferometric systems provided herein may include a processor, such as a central processing unit (“CPU”), a graphics processing unit (“GPU”), or both. Moreover, the system can include a main memory and a static memory that can communicate with each other via a bus. Additionally, the system may include one or more input devices, such as a keyboard, touchpad, tactile button pad, scanner, digital camera or audio input device, and a cursor control device such as a mouse. The interferometric system can include a signal generation device, such as a speaker or remote control, and a network interface device.

According to one embodiment, the interferometric system may include color indication means to provide a visible color change to identify a particular analyte. According to one embodiment, the system may include a reference component that provides secondary confirmation that the system is working properly. Such secondary confirmation may include a visual confirmation or analyte reference that is detected and measured by the detector.

The interferometric system as provided herein may also include a transmitting component. The transmitting component may be in electronic signal communication with the detector component. The transmitting component sends or transmits a signal regarding analyte detection and quantification data. The transmission of such data may include real-time transmission via any of a number of known communication channels, including packet data networks and in any of a number of forms, including text messages, email, and so forth. Such real-time transmission may be sent to a remote destination via a wireless signal. The wireless signal may travel via access to the Internet via a surrounding Wi-Fi network. The wireless signal may also communicate with a remote destination via Bluetooth or other radio frequency transmission. The remote destination may be a smart phone, pad, computer, cloud device, or server. The server may store any data for further analysis and later retrieval. The server may analyze any incoming data using artificial intelligence learning algorithms or specialized pathological, physical, or quantum mechanical expertise programed into the server and transmit a signal.

According to one embodiment, the interferometric system includes a wireless data link to a phone line. Alternatively, a wireless data link to a building Local Area Network may be used. The system may also be linked to Telephone Base Unit (TBU) which is designed to physically connect to a phone jack and to provide 900 MHz wireless communications thereby allowing the system to communicate at any time the phone line is available.

According to one embodiment, the system may also include geolocation information in its communications with the server, either from a GPS sensor included in the system or a GPS software function capable of generating the location of the system in cooperation with a cellular or other communication network in communication with the system. According to a particular embodiment, the system may include a geolocation device (such as GPS or RFID) either from within its own device or from a mobile phone or similarly collocated device or network to determine the physical location of the system.

According to one embodiment, the interferometric system includes an external camera. The external camera may be at least partially located within the interferometric system housing but include a lens exposed to the exterior of the housing such that the external camera may take photos and video of a target sample prior to collection (e.g., process fluid, soil, plant, etc.). The external camera may capture video or images that aid in the identification of an analyte and confirmation of the resulting data. The external camera may also capture video images that aid in selecting a proper remedial measure. The external camera may capture video or images that aid in the identification of a target sample or source thereof.

The external camera may capture video or images in connection with scanning and identifying a QR code (such as a QR code on an external surface of a cartridge housing). When located on an external surface of the cartridge housing, the QR code may also aid in identifying ownership of generated data and transmission of such data to a correct owner.

According to one embodiment, the cartridge system contains a geo-location capability that is activated when a sample is analyzed to “geo-stamp” the sample results for archival purposes. According to one embodiment, the cartridge system contains a time and date capability that is activated when a sample is analyzed to time stamp the sample results for archival purposes. According to one embodiment, the cartridge system includes materials that are biodegradable, or recycled materials, to reduce environmental impact. Any used cartridge system provided herein may be disposed of in any acceptable manner such as via a standard biohazard container. According to one embodiment, the cartridge system facilitates cleaning and re-tooling to allow the cartridge system to be replenished and returned to operation.

According to one embodiment, the cartridge system is stabilized to address vibrational distortions. The system may be stabilized by various stabilization means including mechanical (alignment means as provided herein), chemically (fluid float or gel pack), computer-assisted system (electronically), or digitally (e.g., via a camera or digital processing).

### Target Sample Handling System

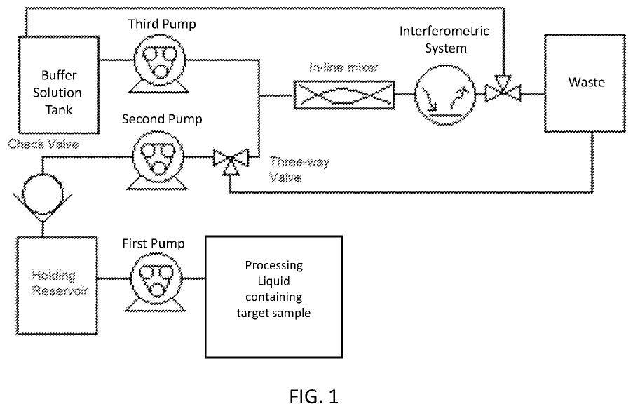

A target sample handling system may be utilized in conjunction with the interferometric systems provided herein as illustrated in FIG. 1. The target sample handling system may utilize a flow injection system in fluid communication with an interferometric for batch or continuous in-line or in situ sensing, monitoring and quantification of one or more analytes such as peracetic acid.

The flow injection system may be configured to pull processing liquid (e.g., processing water) from a liquid source believed to include target sample. The liquid source of target sample may be optionally in liquid communication with a first pump for moving target sample to a holding reservoir. The holding reservoir may be optionally in liquid communication with a check valve to prevent backflow of target sample liquid. The check valve may be optionally in liquid communication a second pump that is, in turn, in liquid communication with a three-way valve. The three-way valve is suited to alternate between alternate between target sample liquid, buffer, and waste.

The flow injection system may further include an in-line mixer in liquid communication with a third pump that is, in turn, in liquid communication with a buffer tank for holding liquid buffer. The in-line mixer is suited for mixing the target sample liquid with buffer to form a test sample composition. The in-line mixer may be in liquid communication with an interferometric system as provided herein that includes a sensing layer composition. After passing through the interferometric system, the test sample composition may move to waste for disposal.

According to one embodiment, the flow injection system can be programed for any target sample flow rate and contact time with the sensing layer composition. According to one embodiment, the frequency of peracetic acid sensing and quantification may be continuous. According to one embodiment, the frequency of peracetic acid sensing and quantification may be at regular intervals. Such intervals may be custom defined and adjusted. Such intervals may be, for example, every 30 seconds, once per minute, twice per hour, once per hour, every hour or any other interval deemed suitable by a user.

According to one embodiment, an open-source electronic platform such as Arduino™ may be used to provide the controls for pumps and valves. According to one embodiment, a computer and associated processing equipment such as Jetson Nano™ may be used to collect and process the sensing and quantification data. According to one embodiment, the target sample handling system and interferometric systems may be housed in a wash-down enclosure to provide water resistance to all system components. The wash-down enclosure may be movable or otherwise portable.

### Sensing Layer Composition

A sensing layer composition is provided. The sensing layer composition is sensitive to and may selectively bind to one or more analytes. The sensing layer composition may also reversibly release one or more analytes. The sensing layer composition may be adhered to at least one side of one or more waveguide channels in/on a waveguide chip of an interferometric system. According to a particular embodiment, the sensing layer composition is adapted to bind one or more analytes that are detected via interferometric analysis.

According to a particular embodiment, the sensing layer composition is configured to selectively bind one or more analytes in a liquid buffer solution. According to a particular embodiment, the sensing layer composition is configured to selectively bind one or more analytes in a buffer solution that includes acetic acid and hydrogen peroxide without interference from the acetic acid and hydrogen peroxide. According to one embodiment, the buffer solution may include potassium iodide in an amount of from about 0.001% w/w to about 10.0% w/w. According to one embodiment, the buffer solution may include potassium iodide in an amount of about 0.01% w/w.

According to one embodiment, the sensing layer composition is a charge-transfer based sensing layer composition. According to one embodiment, the sensing layer composition is formulated as a charge-transfer based sensing film. According to one embodiment, the sensing layer composition is iodine based. According to one embodiment, the sensing layer composition is formulated as an iodine based film. According to one embodiment, the sensing layer composition includes a charge transfer complex formed between iodine and at least one aromatic hydrocarbon such an aromatic polymer. Aromatic polymers containing electron donating functional groups include, but are not limited to, phenol and styrene.

According to one embodiment, the charge transfer complex may be dissociated so the sensing layer composition can be used more than one time (i.e., used to selectively bind to and reversibly release one or more analytes. According to one embodiment, the sensing layer composition is configured to sense peracetic acid in a liquid carrier in conjunction with an optical interferometer as provided herein that is configured to detect or sense peracetic acid.

According to one embodiment, the sensing layer composition is configured to sense and quantify peracetic acid at levels of parts per million (ppm), parts per billion (ppb) or parts per trillion (ppt) level. According to one embodiment, the sensing layer composition may include an automated flow injection system that is configured to collect chiller water, conduct in situ dilution, introduce a buffer solution to the target sample, and deliver a diluted test sample composition stream to a flow cell.

### Methods of Use

According to one embodiment, a fluid source of analytes includes an industrial or commercial vessel adapted to store, process, or carry one or more chemicals that may contain peracetic acid. Such a vessel may be located within or around a shipping container that stores and transports a fluid chemical. The shipping container may be located on a truck, train, or other means of transportation. The shipping container may also be located on or around shipping dock.

According to a particular embodiment, the systems and methods provided herein may be utilized to detect and quantify levels of a peracetic in an industrial environment such as in a chemical processing or chemical manufacturing facility. By providing detection and quantification data in an efficient manner within the production environment, exposure to peracetic acid may be monitored, adjusted and otherwise controlled. According to such an embodiment, the system will detect and quantify peracetic acid at the parts per million (ppm), parts per billion (ppb) or parts per trillion (ppt) level.

According to a particular embodiment, the systems and methods provided herein may be utilized to detect, quantify or otherwise monitor levels of peracetic acid in a variety of other applications in various environments. Such applications include food processing facilities, healthcare facilities, manufacturing or processing facilities (e.g., pulp/paper manufacturing facilities), water treatment plants (municipal or bottled), laboratories, wineries/breweries or laundry facilities. Other application include waste management, national defense, metal coating, plastics/resins, mining and refining, airports, petroleum extraction and refining, paints/coatings, metal machinery manufacturing, packaging production, disposable plate production, electronics, oil/gas, printing, textiles/leathers, cleaning products manufacturing, glass products, cement manufacturing, and home accessories/building products (e.g., carpet, furniture).

According to one particular embodiment, the interferometric system provided herein may be utilized in connection with or otherwise equipped to a mobile vehicle. Suitable mobile vehicles include, but are not limited to, unmanned aerial vehicles (UAV), unmanned ground vehicles (UGV), drones, manned aircraft, and manned vehicles.

According to one particular embodiment, the interferometric system provided herein may be utilized in connection with or otherwise equipped to a water supply system to continuously monitor (or batch monitor) the water for peracetic acid. According to one particular embodiment, the interferometric system provided herein may be connected to a water faucet in a variety of locations such as in a home, laboratory or industrial setting.

### Methods of Detection and Quantification

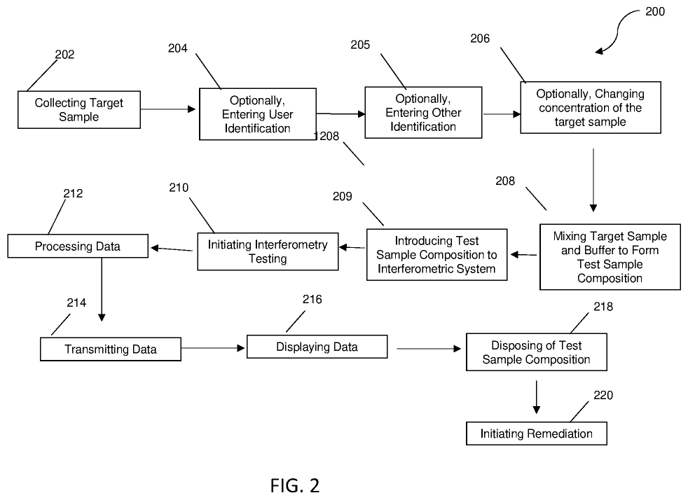

FIG. 2 illustrates a method 200 of detecting and quantifying the level of one or more analytes such as peracetic acid according to one embodiment. The method includes the step of collecting 202 or otherwise obtaining a target sample having one or more analytes such as peracetic aid. In different embodiments, the target sample may be taken from the appropriate target depending on the location and environment. A target sample handing system may be utilized to move target sample through one or more valves or pumps prior to the next step.

According to one embodiment, the method further includes the optional step of entering 204 a user identifier (ID) in the interferometric system. Additionally, an identification number associated with the sample, analyte or interest or a combination thereof may be entered. The cartridge system utilized may be equipped with a label or sticker carrying identifying such information.

According to one embodiment, the method further includes the optional step of entering other information 205. The label or sticker may include a QR code including such information. The label or sticker may be removed prior to use. Identifying information may include metadata such as time, GPS data, or other data generated by the interferometric system.

According to one embodiment, the method further includes the optional step 206 of changing the concentration of the target sample. According to one embodiment, the optional step 206 of changing the concentration of the target sample include dilution of the target sample. According to one embodiment, the optional step 206 of changing the concentration of the target sample is carried out by solid-solid extraction/solid phase extraction (SPE), microbeads, or other suitable means for concentrating, purifying or otherwise preparing the test sample for detection and quantification.

According to one embodiment, the method further includes the step of mixing 208 the target sample with a buffer solution to form a test sample composition. According to one embodiment, the buffer solution is aqueous based. According to one embodiment, the buffer solution includes an acetate buffer. According to one embodiment, the buffer solution may include potassium iodide. In a multiple-use cartridge system, mixing 208 the target sample with a buffer solution to form a test sample composition may occur prior to the test sample composition being introduced to the cartridge system such as by an in-line mixer. Optionally, the steps 206 and 208 may be combined into a single step.

According to one embodiment, the method further includes the step of introducing the test sample composition to the interferometric system 209. According to one embodiment, target sample composition is introduced to the cartridge by one or more components of the target sample handling system provided herein. According to one embodiment, target sample composition is introduced to the cartridge by a separate device such as a syringe or pump. According to one embodiment, target sample is introduced by an injection device. According to one embodiment, the injection device may be permanently attached to the cartridge system. According to one embodiment, the injection device is a pipette. According to one embodiment, the injection device is a syringe. According to one embodiment, the injection device is a lance, pipette or capillary tube. When utilizing a multiple-use cartridge system, the cartridge system may be fitted to a tube or other transfer mechanism to allow the sample to be continuously taken from a large amount of fluid that is being monitored.

The method of detecting and quantifying the level of analyte in a sample includes initiating waveguide interferometry 210 on the test sample. Such a step may include initiating movement of the light signal through the cartridge system as provided herein and receiving the light signal within the detector unit. Any changes in an interference pattern are representative of analyte in the test sample. Particularly, such changes in an interference pattern generate data related to one or more analyte in the test sample composition. According to one embodiment, the step of initiating 210 waveguide interferometry on the test sample composition includes the step of correlating data from the phase shift with calibration data to obtain data related to analyte identity, analyte concentration, or a combination thereof.

According to one embodiment, the method further includes the step of processing 212 any data resulting from changes in the interference pattern. Such changes in interference pattern may be processed and otherwise translated to indicate the presence and amount of an analyte in a test sample. Processing may be assisted by software, processing units, processor, servers, or other component suitable for processing. The step of processing data may further include storing such data in a storage means.

According to one embodiment, the method optionally includes the step of transmitting a data signal 214. The signal may result in the displaying of data on the system 216. The step of transmitting data may include displaying the analyte levels via projecting any real time data on a screen as described herein. The step of transmitting data may include transmitting any obtained data to a mobile phone, smart phone, tablet, computer, laptop, watch or other process control device. The data may also be sent to a device at a remote destination. The remote destination device may be a locally operated mobile or portable device, such as a smart phone, tablet device, pad, or laptop computer. The destination may also be smart phone, pad, computer, cloud device, or server. In other embodiments, the remote destination may be a stand-alone or networked computer, cloud device, or server accessible via a local portable device.

The method may optionally include the step of disposing of the test sample 218 per legal requirements. Such legal requirements assure that any sample still containing unacceptable levels of analyte such as peracetic aid is disposed of properly so as not to cause harm to a user or the environment.

According to one embodiment, the method optionally includes the step of initiating 220 a cleaning or remedial countermeasure against any analyte detected.

Although the present specification describes components and functions that may be implemented in particular embodiments with reference to particular standards and protocols, the invention is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be apparent to those skilled in the art, and all modifications that do not depart from the spirit of the disclosure are intended to be included with the scope of the appended claims.

### Example 1

### Sensing Composition Field Test and Validation

### Peracetic Acid Detection

The sensing layer composition provided herein was employed in an interferometer (optical waveguide) to test and validate the sensing layer composition's ability to sense and select peracetic acid. Specifically, an optical interferometer having waveguide channels coated with the sensing layer composition provided herein was installed next to a poultry chiller water tank inside a poultry processing plant. The water from the chiller was tested for peracetic acid via titration, N,N-diethyl-p-phenylelnediamine (DPD), and gas chromatography/mass spectrometry (GC/MS) for comparison to peracetic acid detection and quantification data obtained with the interferometer containing the sensing layer composition.

All methods of measurement showed similar peracetic acid quantity (ppm) fluctuation daily. Although similar trend on PAA variations were obtained from all three methods, titration based method produced highest value while the sensor based measurement gave the lowest values. One-way Anova was performed and determined there was no significant difference between peracetic acid quantification data obtained via GC/MS and optical interferometry with the sensing layer composition.

### GENERAL STATEMENTS

The following statements provide a general description of the disclosure and are not intended to limit the appended claims.

Statement 1. A sensing layer composition is provided that includes a charge transfer complex comprising an electron acceptor and at least one aromatic hydrocarbon, wherein the sensing layer composition is adapted to bind or otherwise be selectively disturbed by one or more analytes.

Statement 2. The sensing layer composition of statement 1, wherein the electron acceptor comprises iodine and the at least one aromatic hydrocarbon comprises an aromatic polymer.

Statement 3. The sensing layer compositions of statements 1-2, wherein the aromatic polymer comprises phenol, styrene, or a combination thereof.

Statement 4. The sensing layer compositions of statements 1-3, wherein the sensing layer composition is configured to sense one or more analytes in a liquid that contains acetic acid or hydrogen peroxide without interference from the acetic acid or the hydrogen peroxide.

Statement 5. The sensing layer compositions of statements 1-4, wherein the sensing layer composition is adapted to be adhered to at least one side of one or more waveguide channels in/on a waveguide chip of an interferometric system.

Statement 6. The sensing layer compositions of statements 1-5, wherein the interferometric system is an optical interferometric system.

Statement 7. The sensing layer compositions of statements 1-6, wherein the sensing layer composition is formulated as a film.

Statement 8. The sensing layer compositions of statements 1-7, wherein the one or more analytes includes peracetic acid.

Statement 9. An analyst sensor system is provided that includes a target sample handing system; and an interferometric system, wherein the sensor system is configured to detect and quantify one or more analytes present in a target sample, and wherein the target sample handling system and interferometric system are in liquid communication with one another.

Statement 10. The analyte sensor of statement 9, wherein the at least one analyte is peracetic acid.

Statement 11. The analyte sensor system of statements 9-10, wherein the target sample handing system includes a flow injection system including at least one pump; and at least one in-line mixer.

Statement 12. The analyte sensor system of statements 9-11, configured to detect and quantify one or more analytes in situ and provide analyte quantity in real-time or near real-time.

Statement 13. The analyte sensor system of statements 9-12, wherein the interferometric system is an optical interferometric system that includes a sensing layer composition including a charge transfer complex comprising an electron acceptor and at least one aromatic hydrocarbon, wherein the sensing layer composition is adapted to bind or otherwise be selectively disturbed by one or more analytes.

Statement 14. The analyte sensor system of statements 9-13, wherein the sensing layer composition is formulated as a film.

Statement 15. The analyte sensor system of statements 9-14, wherein the one or more analytes includes peracetic acid.

Statement 16. A method of detecting and quantifying the level of analyte in a target sample, the method includes the steps of:
collecting a target sample containing one or more analytes;
optionally entering an identification associated with the target sample;
optionally, concentrating the target sample;
optionally, mixing the target sample with a buffer solution to form a test sample composition;
introducing the target sample to an interferometer;
initiating waveguide interferometry on the target sample;
processing any data resulting from the waveguide interferometry; and
optionally, transmitting any data resulting from the waveguide interferometry,
wherein the interferometer includes an interferometric chip that, in turn, includes one or more waveguide channels having a sensing layer composition thereon, the sensing layer composition including a charge transfer complex comprising an electron acceptor and at least one aromatic hydrocarbon, the sensing layer composition adapted to bind or otherwise be selectively disturbed by one or more analytes within the target sample.

Statement 17. The method of statement 16, wherein the step of transmitting data includes wirelessly transmitting analyte detection and quantification data to a mobile device or server.

Statement 18. The method of statements 16-17, further comprising the step of displaying data related to the presence of analyte in the target sample on a display unit.

Statement 19. The method of statement 16-18, wherein the target sample is taken from water, soil or air.

Statement 20. The method of statement 16-19, wherein the data resulting from the interferometry is provided at or under 30 minutes.

We claim:

1. A sensing layer composition comprising:
a charge transfer complex comprising an electron acceptor and at least one aromatic hydrocarbon,
wherein the sensing layer composition is adapted to bind or otherwise be selectively disturbed by one or more analytes.

2. The sensing layer composition of claim 1, wherein the electron acceptor comprises iodine and the at least one aromatic hydrocarbon comprises an aromatic polymer.

3. The sensing layer composition of claim 2, wherein the aromatic polymer comprises phenol, styrene, or a combination thereof.

4. The sensing layer composition of claim 1, wherein the sensing layer composition is configured to sense one or more analytes in a liquid that contains acetic acid or hydrogen peroxide without interference from the acetic acid or the hydrogen peroxide.

5. The sensing layer composition of claim 1, wherein the sensing layer composition is adapted to be adhered to at least one side of one or more waveguide channels in/on a waveguide chip of an interferometric system.

6. The sensing layer composition of claim 5, wherein the interferometric system is an optical interferometric system.

7. The sensing layer composition of claim 1, wherein the sensing layer composition is formulated as a film.

8. The sensing layer composition of claim 1, wherein the one or more analytes includes peracetic acid.

9. An analyst sensor system comprising
a target sample handing system; and
an interferometric system,
wherein the sensor system is configured to detect and quantify one or more analytes present in a target sample, and
wherein the target sample handling system and interferometric system are in liquid communication with one another.

10. The analyte sensor system of claim 9, wherein the at least one analyte is peracetic acid.

11. The analyte sensor system of claim 9, wherein the target sample handing system includes a flow injection system comprising:
at least one pump; and
at least one in-line mixer.

12. The analyte sensor system of claim 9, configured to detect and quantify one or more analytes in situ and provide analyte quantity in real-time or near real-time.

13. The analyte sensor system of claim 9, wherein the interferometric system is an optical interferometric system that includes a sensing layer composition comprising:
a charge transfer complex comprising an electron acceptor and at least one aromatic hydrocarbon,
wherein the sensing layer composition is adapted to bind or otherwise be selectively disturbed by one or more analytes.

14. The analyte sensor system of claim 13, wherein the sensing layer composition is formulated as a film.

15. The analyte sensor system of claim 9, wherein the one or more analytes includes peracetic acid.
